(19) United States
(12) Patent Application Publication
(10) Pub. No.: US 2020/0407479 A1
(43) Pub. Date: Dec. 31, 2020

(54) POLYETHER-BASED BLOCK COPOLYMERS HAVING HYDROPHOBIC DOMAINS

(71) Applicant: Johannes Gutenberg-Universität Mainz, Mainz (DE)

(72) Inventors: Patrick VERKOYEN, Mainz (DE); Jan BLANKENBURG, Mainz (DE); Holger FREY, Emmendingen (DE)

(73) Assignee: Johannes Gutenberg-Universität Mainz, Mainz (DE)

(21) Appl. No.: 16/976,638

(22) PCT Filed: Mar. 27, 2019
(86) PCT No.: PCT/EP2019/057727

§ 371 Date: Aug. 28, 2020

(30) Foreign Application Priority Data
Mar. 28, 2018 (DE) 102018107464.6

(51) Int. Cl. C08F 293/00; A61K 47/32; A61K 47/34

(52) U.S. Cl. CPC C08F 293/00; A61K 47/34; A61K 47/32

(57) ABSTRACT

The invention relates to a polymerization method in which alkyl glycidyl ethers and epoxides, such as ethylene oxide, polypropylene oxide, 1-ethoxyethyl glycidyl ether and gycidol, are copolymerized and block copolymers are synthesized. The inventive methods include an initiator, oligomer blocks of 1 to 40 alkyl glycidyl ether units of type (I), (II) or (III), and 80 to 1000 epoxy units of large polyether blocks, such as polyethylene oxide (PEO), polypropylene oxide (PPO), polyethoxyethylene glycidyl ether (PEEGE), linear and branched polyglycidol (PG, hbPG) or random copolymers of two, three or four different epoxide units, such as ethylene oxide (EO), propylene oxide (PO), 1-ethoxyethyl glycidyl ether (EEGE) and/or glycidol.

The present invention relates to hydrogels composed of amphiphilic block copolyethers having an AB multiblock structure, in which the A blocks are formed by alkyl glycidyl ethers and the B blocks consist of polyethers such as polyethylene oxide (PEO), polypropylene oxide (PPO), polyethoxyethylene glycidyl ether (PEEGE), linear or branched polyglycidol (PG, hbPG) or random copolymers of ethylene oxide (EO), propylene oxide (PO), 1-ethoxyethyl glycidyl ether (EEGE) and/or glycidol, and also a process for the production thereof. The hydrogels or amphiphilic block copolyethers are used, inter alia, as drug delivery systems in medicine and also in industrial applications as materials having high mechanical damping.

In the prior art, aliphatic polyethers produced by ring opening polymerization (ROP) of the epoxide monomers ethylene oxide (EO), propylene oxide (PO) and, to a lesser extent, butylene oxide (BO) form an established and important class of polymers which are utilized commercially for numerous applications. The characteristic properties of materials based on polyethers are due to their backbone, in particular their high flexibility, which leads to low glass transitions at temperatures of ≤−60° C., and their hydrophilicity due to the C—O—C bond. These properties are not possessed by polymers having a backbone based on carbon, e.g. polyolefins or other vinyl polymers. Although three- to five-membered cyclic ethers can generally be polymerized in order to produce polyethers by ROP, epoxides are clearly the most versatile class of monomers for the synthesis of polyethers since they can be polymerized by various mechanisms and EO and PO are produced industrially in large amounts by direct oxidation of the respective alkenes. The driving force for ROP is the high ring strain of epoxides, which in the case of ethylene oxide is in the order of 110-115 kJ/mol. This allows polymerization of epoxide monomers (IUPAC: oxirane) in three ways: (i) by means of a base, (ii) acid-initiated and (iii) by coordination polymerization. Other classes of epoxide monomers, e.g. epichlorohydrin, relatively long-chain alkylene epoxides, various glycidyl ethers and glycidyl amines, play an increasingly important role in medical and industrial applications and open up highly promising opportunities. The structurally simple key monomers, which are shown in scheme 1, are produced globally in a quantity of more than 33 million metric tons per year.

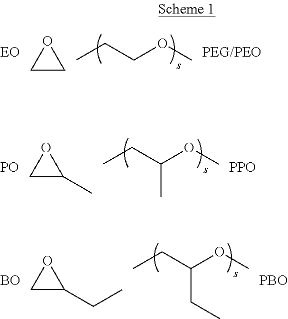

As early as 1863, Wurtz reported the polymerization of EO in the presence of alkali metal hydroxide or zinc chloride. In 1929, Staudinger and Schweitzer developed synthetic methods for a series of model polymers based on EO and classified these on the basis of detailed viscosimetric studies according to their properties, in particular their molecular weight. In the 1930s, poly(ethylene glycol) produced by means of addition of EO onto ethylene glycol under basic conditions was brought onto the market. In the following decade, PEG was used in pharmaceuticals, lubricants, cosmetics and laundry detergents.

In a pioneering study in 1940, Flory described the mechanism of the base-initiated polymerization of EO as living chain growth, which resulted in a molecular weight having a Poisson distribution.

In the 1940s, liquid polyols produced by means of anionic polymerization of propylene oxide were utilized for hydraulic fluids and lubricants. The use of PEO as polar, water-soluble block for nonionic surfactants was a further important development in the 1950s. The corresponding fatty alcohol ethoxylates and alkylphenol ethoxylates are nowadays produced on a scale of millions of metric tons and represent the most important class of nonionic surfactants. Poly(ethylene glycol) is an important biocompatible standard polymer for pharmaceutical, cosmetic and medical applications and is used in numerous products such as skincare formulations, tablet formulations, laxatives and food additives. EO polymers having a high molecular weight are usually referred to as poly(ethylene oxide), PEO for short, and sometimes as poly(oxyethylene), POE for short. On the other hand, the term poly(ethylene glycol), PEG for short, has become established for polymers having molecular weights below 30 000 g/mol. In medical applications, the term “PEG” is generally used. The abbreviation mPEG refers to a monomethyl ether-terminated PEG having a single terminal hydroxyl group which can be further functionalized with PEG for the block copolymer synthesis or bioconjugation, usually referred to as “PEGylation”. PEG is a crystalline thermoplastic polyether which is very readily water-soluble in virtually all concentrations and has a very low immunogenicity, antigenicity and toxicity. The high water solubility of PEG is unique among aliphatic polyethers which are usually insoluble in water. The water solubility can be attributed to the spacing of the oxygen atoms in the polymer structure, which corresponds to the spacing of the hydrogen atoms in a water molecule. PEG and PEO are liquids or low-melting solids. The melting point of PEG depends on the chain length and in the case of PEO having a high molecular weight is 65° C. For skin creams, ointments and suppositories, the melting point is set in the region of the body temperature by mixing and cocrystallization of PEG of various molecular weights. PEG is available under various trade names, for example Carbowax™, Polyox™, Macrogol, Fortrans, Pegoxol.

In biochemical medicine, PEG is used for modifying therapeutic molecules and active substance carriers such as peptides or proteins and liposomes, in order to increase the physiological retention time. This “PEGylation” gives the active substance molecule or carrier “stealth properties” and has in recent years found numerous applications in pharmacy and biochemistry. More recent developments in PEGylation relate to nonionic, amphiphilic block copolymers of PEG with effectively nonpolar polypropylene oxide.

Poly(propylene oxide), PPO for short, in the case of a low molecular weight also referred to as poly(propylene glycol) or PPG, is produced by means of ROP of propylene oxide (PO). PPO is a flexible polymer (glass transition temperature −70° C.) which is formed from racemic PO monomer, has a nonstereoregular (atactic) structure and is therefore not crystalline. In contrast to PEG or PEO, PPO is not water-soluble at room temperature because of the additional methyl group in every repeating unit, which sterically shields the framework. On the other hand, PPG having a low molecular weight is soluble in aqueous solvents at low temperature, with the lower critical dissolution temperature being about 15° C., depending on the molecular weight.

The industrial base-initiated polymerization of PO is based mainly on potassium hydroxide and alcohols as initiators. Since a considerable proportion of PO is used for the production of star polymers, polyols such as glycerol, pentaerythritol or sorbitol are often used as multifunctional initiators. PP star polyether polyols play a key role in the synthesis of flexible polyurethane foams because of their chain flexibility, i.e. a low glass transition temperature, and their amorphous nature. In general, PPG is used for lubricants, antifoaming agents, plasticizers, rheological modifiers, flexible polyamide (urethane) foams and nonionic surfactants, often in combination with PEG.

In contrast to EO and PO, 1,2-butylene oxide monomer, hereinafter referred to as butylene oxide (BO), is produced in a two-stage industrial process and cannot be obtained by direct oxidation of the corresponding alkene. The properties of poly(butylene oxide) (PBO) resemble those of PPO, but it is, as expected, more hydrophobic. The high hydrophobicity of PBO is advantageous for various applications, for example for polyurethanes which have to be stable to water or hot steam. Small amounts of PBO which are added to lubricants can improve the properties of the latter. In some cases, BO is used as comonomer in order to modify the properties of other polyethers, i.e. in order to increase their nonpolar and amorphous structure. The increased hydrophobicity of PBO is an advantage for surfactants which combine PEO and PBO blocks.

Since the 1930s, the oxyanionic polymerization of epoxides has been the standard method for the polymerization of polyethers and is still today used for the major part of the PEO and PPO produced industrially, even though it has various disadvantages, in particular a restriction of the molecular weight of PPO. The anionic polymerization of EO is based on nucleophiles as initiators. The standard method for the industrial synthesis of low molecular weight PEG is the controlled addition of EO onto water or alcohols as initiators in the presence of alkaline catalysts. In most cases, alkali metal compounds having a high nucleophilicity are used. To achieve relatively high molecular weights, alkali metal hydrides, alkyls, aryls, hydroxides, alkoxides and amides can be used for the living anionic polymerization of EO in an inert solvent. As in the case of all ionic polymerizations, the counterion plays a key role and should have a low Lewis acidity and preferably a low interaction, or no interaction, with the chain end. Solvents used for the anionic polymerization of epoxides have to be polar and aprotic. For this reason, tetrahydrofuran (THF), dioxane, dimethyl sulfoxide (DMSO) and hexamethylphosphoramide (HMPA) are often used. In addition, polymerization in the bulk monomer is possible when low molecular weights are sought, but with an increased polydispersity. The fundamentals of these established processes have been well known since the late 1980s. Alkoxides having sodium, potassium or cesium counterions in ethers (usually THF) or other polar, aprotic solvents are the most widely used initiator systems. The addition of complexing agents such as crown ethers which bind the respective cation can greatly accelerate epoxide polymerization. Since the polymerization of epoxides is a living process, a Poisson distribution is obtained, which allows simple and quantitative end functionalization of the resulting PEG. The active alkoxide chain end of the growing PEG is relatively stable and the mechanism of the polymerization is simple (Scheme 2).

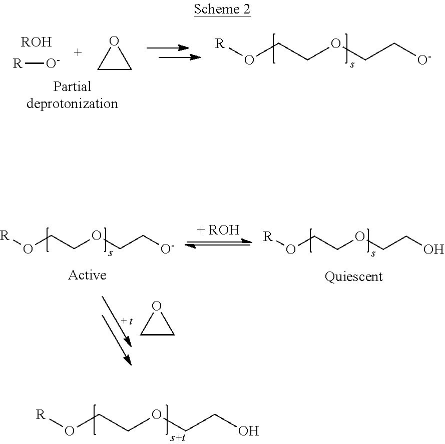

Owing to the fast proton exchange equilibrium, partial deprotonation of the alkoxide initiator (often only 10-20%) is sufficient. In such synthesis methods, the chain growth can be considered to be polymerization with degenerative chain transfer, i.e. a reversible end, with the hydroxyl terminus of the chain forming the quiescent species and the alkoxide end forming the active species. Since the proton exchange occurs very rapidly, a combination of an alkoxide with the corresponding alcohol is in most cases used as initiator system. This applies particularly to the synthesis of polyether polyols (i.e. PPO or PPO/PEG star polymers), in order to retain the solubility of the respective multihydroxy-functional initiator.

Apart from alkoxides, it is also possible to use strongly nucleophilic hydrides, amides and alkyl or aryl compounds of sodium, potassium and cesium in order to initiate the polymerization of EO. The oxyanionic polymerization of EO in solution is based on the oxygen atom at the charged end of the growing chain as active site at which the negative charge is localized. Depending on the counterion, solvated contact ion pairs can be present. In addition, the active chain ends themselves can be strongly associated in dilute solution. The presence and reactivity of aggregated species and ion pairs compared to free ions and their respective contributions to oxyanionic polymerization of EO (cf. Scheme 3) can be observed for a contact ion pair in a sodium methoxide-initiated polymerization of EO.

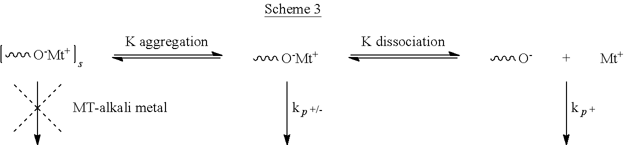

The reaction rate of the alkoxide-initiated polymerization is usually slow, but can be accelerated to a certain extent by increasing the temperature and also by means of a small excess of the corresponding alcohol. This is explained by the formation of an initiator complex of alcohol and alkoxide, which leads to a partial separation of the ion pair at the end of the chain. The relatively low rate of the EO polymerization in various solvents allows in-situ measurement by means of NMR spectroscopy, with the monomer sequence of the polymer chain being able to be determined directly. The terminal functionalization of PEG can be examined and optimized by means of MALDI-TOF spectroscopy. Therefore the mechanism of oxyanionic EO polymerization is well understood in detail. A particular feature of the synthesis of PEG by anionic polymerization is the participation of the oxygen atoms of the polyether main chain in the solvation of the cation of the ion pair. The mobility of the PEG segments in combination with their solvating action leads to formation of ion triplets and to self-solvation. It is thus possible to observe a “penultimate effect”, i.e. the activity of the chain ends depends on the number of EO units which have already been added on (Scheme 4).

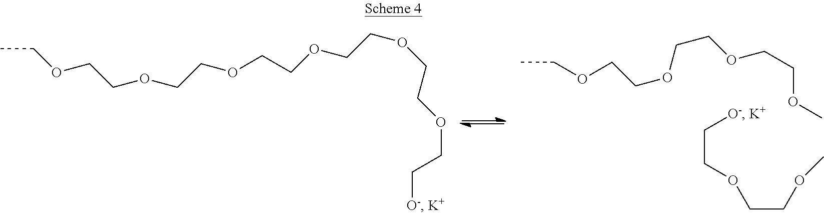

The activation energy for the EO addition at the growing end is 74.5 kJ mol⁻¹. This and the insensitivity of the propagation rate to the type of solvent is explained by the self-associated “shielding effect” of monomer units which are located in the vicinity of the alkoxide chain end. In addition, the interaction between the cation used and the EO monomer can also play a role. A further important feature of the EO polymerization process is a strong temperature dependence. Here, the monomer EO can even be used as inert ether solvent for the anionic polymerization of MMA and 2-vinylpyridine at very low temperatures, which explains its stable character for the carbanionic low-temperature polymerization. The concentration of free ions and associated species can be determined by conductivity measurements. The rate of chain growth in EO polymerization is virtually independent of the solvent used. The oxyanionic polymerization of EO is characterized by (i) tight ion pairs with low dissociation constants (10⁻⁸-10⁻¹² mol l⁻¹) in THF; (ii) the presence of ion triplets and higher associates; (iii) competitive interaction of the growing chains with monomer unit sequences and the EO monomer. This complex nature of the active site represents a fundamental problem in the anionic polymerization of EO, and also for PO and other oxiranes in solution. Accordingly, the molecular weight is restricted to an upper limit of 50000 g/mol. To achieve effective polymerization, preference is given to primary alkoxides since they have a higher reactivity than secondary alkoxides. The polymerization rate of EO is considerably greater than that of PO and critically influences the anionic copolymerization of EO and PO. In general, the reactivity of alkylene oxides decreases with increasing length and bulkiness of the alkyl substituent on the epoxide group. The difficulties associated therewith can be overcome to a certain extent by means of high-temperature and high-pressure polymerization. In addition, the use of potassium or cesium as counterions in ether solution leads to a significantly lower degree of association and consequently to polymerization by the free ions, which likewise improves control of the molecular weight.

The oxyanionic polymerization of propylene oxide is hindered by proton abstraction from the methyl group by the strongly basic initiator system and, associated therewith, extended chain transfer to the PO monomer. The subsequent elimination reaction produces an allyl alkoxide which can induce polymerization of a fresh chain. This leads to PPO having a low molecular weight and an unsaturated allyl end group. Owing to this reaction, the molecular weight of PPO and also of longer alkylene oxides which are produced by oxyanionic polymerization is limited to 6000 g/mol, which is related to the ratio of the polymerization rate constant and the rate constant of the chain transfer to the PO monomer. The counterion influences the chain transfer to the monomer and the resulting isomerization of PO to allyl alcohol. This decreases in the order Na⁺>K⁺>Cs⁺, which is related to the interactions between the metal cation and the alkoxide. Efforts are accordingly being made to develop synthetic methods which keep the living polymerization going for longer and permit higher molecular weights. This is particularly important for the use of PPO as telechelic oligomers and elastomers. The abovementioned secondary reactions can be largely suppressed by means of cesium-initiated systems. Furthermore, the reaction can be inhibited to a certain extent on the transfer side by complexation of the counterion with crown ethers, which likewise contributes to an acceleration of the polymerization. Nevertheless, the molecular weight (Mₙ) of poly(1,2-propylene oxide) (PPO) is limited to 15000 g/mol even in such systems. Thus, a molecular weight of up to 13000 g/mol is achieved in the polymerization in pure propylene oxide at 40° C. using potassium and 18-crown-6 ether as additive.

Polymerization processes as described above are employed in an analogous way in process steps of the present invention.

Hydrogels have long been an interesting class of materials and are used industrially in the coating of implants (B. Jeong, Y. H. Bae, S. W. Kim; Journal of Controlled Release, 1-2, 63, 2000, 155-163; S. B. Goodman, Z. Yan, M. Keeney, F. Yang; Biomaterials, 13, 34, 2013, 3174-3183). Chemical or physical crosslinking of individual polymer chains can create a three-dimensional network which displays swelling behavior resulting from the addition of water. In addition, the swelling behavior of the resulting hydrogel can be influenced by adjustment of the degree of crosslinking (S. K. Shukla, A. W. Sheikh, N. Gunari, A. K. Bajpai, R. A. Kulkarni; Preparation and water sorption study; J. Appl. Polym. Sci., 3, 111, 2009, 1300-1310).

The combination of hydrophilic and hydrophobic block structures gives a broad possibility for modifying the materials properties of the resulting hydrogel (M. Pekar; Front. Mater., 1, 2015, 1003; M. Mihajlovic, M. Staropoli, M. S. Appavou, H. M. Wyss, W. Pyckhout-Hintzen, R. P. Sijbesma; Macromolecules, 8, 50, 2017, 3333-3346). Even in other systems, it has been able to be shown that successful incorporation of hydrophobic active substances into the hydrophobic domains of a hydrogel could be carried out. Thus, for example, the effect of Docetaxel, a leading medicament for the treatment of breast cancer, could be improved significantly by embedding in a hydrogel (Y. Wang, L. Chen, L. Tan, Q. Zhao, F. Luo, Y. Wei, Z. Qian; Biomaterials, 25, 35, 2104, 6972-6985).

Polyethers, in particular PEG and copolymers thereof, are widespread in medical applications and represent the gold standard in medical applications. Owing to their good solubility in water, low toxicity and the “stealth” effect, it is possible to improve the pharmacological and pharmacokinetic properties such as the plasma half life or blood circulation time and at the same time reduce the immunogenicity (C. Dingels, M. Schömer, H. Frey; Chemie in unserer Zeit, 5, 45, 2011, 338-349). Furthermore, it was shown that intramolecular and intermolecular interactions of the hydrophobic units in undefined comb-like copolymers having a random incorporation of hydrophobic compounds (for example long alkyl chains) have an effect on the viscosity in aqueous solution (F. Liu, Y. Frere, J. Francois; J. Polymer, 7, 42, 2001, 2969-2983). A considerable disadvantage of the nonpolar systems described in the prior art is the polymerization process in which very broad molecular weight distributions, which do not meet the regulatory requirements for medical applications, in particular on the part of the Food and Drug Administration of the USA (FDA), are obtained.

In addition, the use of block-type PEG copolymers as additives in order to adjust the viscosity of paints is known in the prior art (K. N. Bakeev et al.; Colorant compatible hydrophobically-modified polyethylene glycol thickener for paint and preparation of water-soluble polymer thickener; US patent application No. 20100324177; 2010).

The present invention provides a novel method for producing amphiphilic copolyethers. It has surprisingly been found that the polymerization of long-chain alkyl glycidyl ethers after addition of a crown ether for complexing the counterion leads to longer blocks of the nonpolar monomer. The method of the invention opens up access to a broad spectrum of long-chain polyalkyl glycidyl ethers and block copolymers having hitherto unachievable molecular weights and a narrow molecular weight distribution or polydispersity PDI≤1.6 to PDI≤1.07.

An important aspect of the block copolyethers of the invention are the hydrophobic A units which consist of long-chain alkyl glycidyl ethers, for example having the chain lengths C₁₂ and C₁₆. Copolymerization of two or more different glycidyl ethers enables the melting point to be set in a targeted manner. This renders use of the hydrogels according to the invention in the medical sector particularly interesting because hydrophobic active substances are bound into the crystalline domains of the hydrogels and can be released by melting at a temperature in the physiological range.

No studies on the synthesis of defined amphiphilic block copolymers of PEG and long-chain alkyl glycidyl ethers, e.g. hexadecyl glycidyl ether and dodecyl glycidyl ether, by means of anionic, ring opening polymerization are known in the prior art. Furthermore, no studies on hydrogels having a melting point of the crystalline hydrophobic domains which can be set in a targeted manner are known.

Owing to their hydrophilic polyether backbone, their well-defined structure and narrow molecular weight distribution (PDI≤1.6 to PDI≤1.07), the block copolyethers of the invention are biocompatible and satisfy the strict requirements for medical approval, in particular on the part of the FDA.

The block copolyethers of the invention form physically crosslinked hydrogels without additives. Furthermore, covalent crosslinking can be brought about by simple chemical modification of the terminal hydroxyl groups or the unsaturated alkyl chains, and the mechanical properties can thus be optimized for the respective application.

Apart from use in the medical sector, the block copolymers of the invention provide a class of materials which have a broad application spectrum and whose mechanical properties can be set in a defined manner. In particular, the melting point of the hydrophobic domains can be varied in a wide range.

It is an object of the present invention to provide a process and a block copolymer synthesized by the process which has a defined molecular weight, viscosity, swelling in water and storage capability for hydrophobic substances.

This object is achieved by a process in which one or more alkyl glycidyl ethers of the type (I), (II), (III)

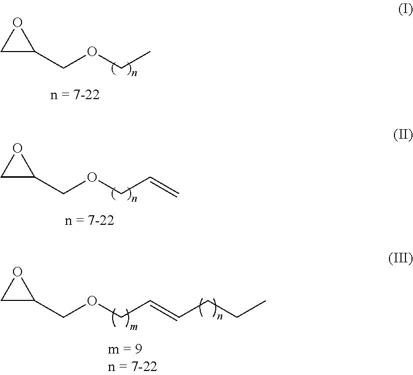

are copolymerized with one or more epoxides selected from the group consisting of ethylene oxide (EO), propylene oxide (PO), 1-ethoxyethyl glycidyl ether (EEGE), glycidol and/or mixtures of two, three or four different epoxides from among these to form blocks composed of polyethylene oxide (PEO), polypropylene oxide (PPO), polyethoxyethylene glycidyl ether (PEEGE), linear or branched polyglycidol (PG, hbPG) and/or random copolymers of the above epoxides.

In addition, the above object is achieved by a process in which one or more alkyl glycidyl ethers of the type (I), (II), (III)

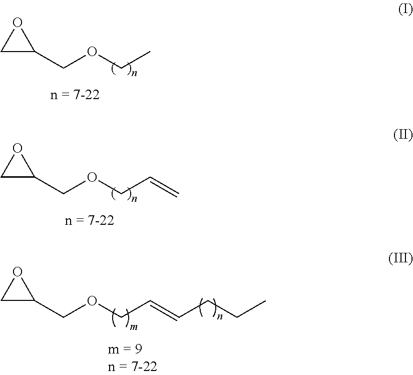

are copolymerized with one or more polyethers selected from the group consisting of polyethylene oxide (PEO), polypropylene oxide (PPO), polyethoxyethylene glycidyl ether (PEEGE), linear or branched polyglycidol (PG, hbPG), monomethyl polyethylene oxide (mPEO), monomethyl propylene oxide (mPPO), monobutyl propylene oxide (mPBO) or a random copolymer of two, three or four different epoxide units such as ethylene oxide (EO), propylene oxide (PO), 1-ethoxyethyl glycidyl ether (EEGE) and/or glycidol.

Advantageous embodiments of the process are characterized in that
7≤n≤12, 10≤n≤15, 12≤n≤17, 15≤n≤20 or 17≤n≤22;
in a first step S₁, a reaction mixture with an initiator I selected from the group consisting of
a deprotonated residual group of an opened alkyl glycidyl ether of the type (I), (II) or (III);
a deprotonated residual group of a polyether such as polyethylene oxide (PEO), polypropylene oxide (PPO), polyethoxyethylene glycidyl ether (PEEGE), linear or branched polyglycidol (PG, hbPG), monomethyl polyethylene oxide (mPEO), monomethyl propylene oxide (mPPO), monobutyl propylene oxide (mPBO) or a random copolymer of two, three or four different epoxide units such as ethylene oxide (EO), propylene oxide (PO), 1-ethoxyethyl glycidyl ether (EEGE) and/or glycidol; and
a deprotonated residual group of an alcohol, for example methanol, butanol, benzyl alcohol (BnOH), 2-(benzyloxy)ethanol, pentaerythritol, 1,1,1-trimethylolpropane (TMP), bisphenol A, CH₃(CH₂)ₜOH and OH(CH₂)ₜOH where t=1-21;
is provided;
in a first step S₁, a reaction mixture with an initiator I which is a deprotonated residual group of a polyether comprising from 80 to 1000 epoxide units, for example polyethylene oxide (PEO), polypropylene oxide (PPO), polyethoxyethylene glycidyl ether (PEEGE), linear or branched polyglycidol (PG, hbPG), monomethyl polyethylene oxide (mPEO), monomethyl propylene oxide (mPPO), monobutyl propylene oxide (mPBO) or a random copolymer of two, three or four different epoxide units, e.g. ethylene oxide (EO), propylene oxide (PO), 1-ethoxyethyl glycidyl ether (EEGE) and/or glycidol, is provided;
in a second step S₂, the initiator I provided in step S₁ is polymerized with from 2 to 40 mol of an alkyl glycidyl ether of the type (I), (II) or (III), a mixture of two or three of the alkyl glycidyl ethers (I), (II), (III) or a mixture of at least one alkyl glycidyl ether (I), (II), (III) with ethylene oxide (EO) and/or 1-ethoxyethyl glycidyl ether (EEGE), based on the molar amount of the initiator I, to give a symmetrical or unsymmetrical oligomer (A₁)_{0.5}I(A₁)_{0.5} or IA₁;
in a second step S₂, the initiator I provided in step S₁ is polymerized with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 mol of an alkyl glycidyl ether of the type (I), (II) or (III), a mixture of two or three of the alkyl glycidyl ethers (I), (II), (III) or a mixture of at least one alkyl glycidyl ether (I), (II), (III) with ethylene oxide (EO) and/or 1-ethoxyethyl glycidyl ether (EEGE), based on the molar amount of the initiator I, to give a symmetrical or unsymmetrical oligomer (A₁)_{0.5}I(A₁)_{0.5} or IA₁;
in a third step S₃, the symmetrical or unsymmetrical oligomer (A₁)_{0.5}I(A₁)_{0.5} or IA₁ obtained in step S₂ is copolymerized with from 80 to 1000 mol of an epoxide, based on the molar amount of the initiator I, to give a symmetrical or unsymmetrical block copolymer (B₁A₁)_{0.5}I(A₁B₁)_{0.5} or IA₁B₁, where the epoxide is selected from the group consisting of ethylene oxide (EO), propylene oxide (PO), 1-ethoxyethyl glycidyl ether (EEGE) and glycidol;
in a third step S₃, the symmetrical or unsymmetrical oligomer (A₁)_{0.5}I(A₁)_{0.5} or IA₁ obtained in step S₂ is copolymerized with a mixture of a total of from 80 to 1000 mol of two, three or four different epoxides, based on the molar amount of the initiator I, to give a symmetrical or unsymmetrical block copolymer (B₁A₁)_{0.5}I(A₁B₁)_{0.5} or IA₁B₁, where the two, three or four epoxides are selected from the group consisting of ethylene oxide (EO), propylene oxide (PO), 1-ethoxyethyl glycidyl ether (EEGE) and glycidol;
in a third step S₃, the symmetrical or unsymmetrical oligomer (A₁)_{0.5}I(A₁)_{0.5} or IA₁ obtained in step S₂ is copolymerized with from 80 to 1000 mol of a first epoxide, based on the molar amount of the initiator I, to give a symmetrical or unsymmetrical block copolymer (B₁A₁)_{0.5}I(A₁B₁)_{0.5} or IA₁B₁ and subsequently with from 80 to 1000 mol of a second epoxide, based on the molar amount of the initiator I, to give a symmetrical or unsymmetrical block copolymer (C₁B₁A₁)_{0.5}I(A₁B₁C₁)_{0.5} or IA₁B₁C₁, where the first and second epoxide are selected from the group consisting of ethylene oxide (EO), propylene oxide (PO), 1-ethoxyethyl glycidyl ether (EEGE) and glycidol and are different from one another;
in a fourth step S₄, the symmetrical or unsymmetrical block copolymer (B₁A₁)_{0.5}I(A₁B₁)_{0.5}, (C₁B₁A₁)_{0.5}I(A₁B₁C₁)_{0.5}, IA₁B₁ or IA₁B₁C₁ obtained in step S₃ is copolymerized with from 2 to 40 mol of an alkyl glycidyl ether of the type (I), (II) or (III) or a mixture of two or three of the alkyl glycidyl ethers (I), (II), (III), based on the molar amount of the initiator I, to give a symmetrical or unsymmetrical block copolymer (A₂B₁A₁)_{0.5}I(A₁B₁A₂)_{0.5}, (A₂C₁B₁A₁)_{0.5}I(A₁B₁C₁A₂)_{0.5}, IA₁B₁A₂ or IA₁B₁C₁A₂;
in a fifth step S₅, the symmetrical or unsymmetrical block copolymer (A₂B₁A₁)_{0.5}I(A₁B₁A₂)_{0.5}, (A₂C₁B₁A₁)_{0.5}I(A₁B₁C₁A₂)_{0.5}, IA₁B₁A₂ or IA₁B₁C₁A₂ obtained in step S₄ is copolymerized with from 80 to 1000 mol of an epoxide, based on the molar amount of the initiator I, to give a symmetrical or unsymmetrical block copolymer (B₂A₂B₁A₁)_{0.5}I(A₁B₁A₂B₂)_{0.5}, (B₂A₂C₁B₁A₁)_{0.5}I(A₁B₁C₁A₂B₂)_{0.5}, IA₁B₁A₂B₂ or IA₁B₁C₁A₂B₂, where the epoxide is selected from the group consisting of ethylene oxide (EO), propylene oxide (PO), 1-ethoxyethyl glycidyl ether (EEGE) and glycidol;
in a fifth step S₅, the symmetrical or unsymmetrical block copolymer (A₂B₁A₁)_{0.5}I(A₁B₁A₂)_{0.5}, (A₂C₁B₁A₁)_{0.5}I(A₁B₁C₁A₂)_{0.5}, IA₁B₁A₂ or IA₁B₁C₁A₂ obtained in step S₄ is copolymerized with a mixture of a total of from 80 to 1000 mol of two, three or four different epoxides, based on the molar amount of the initiator I, to give a symmetrical or unsymmetrical block copolymer (B₂A₂B₁A₁)_{0.5}I(A₁B₁A₂B₂)_{0.5}, (B₂A₂C₁A₁B₁)_{0.5}I(A₁B₁C₁A₂B₂)_{0.5}, IA₁B₁A₂B₂ or IA₁B₁C₁A₂B₂, where the two, three or four epoxides are selected from the group consisting of ethylene oxide (EO), propylene oxide (PO), 1-ethoxyethyl glycidyl ether (EEGE) and glycidol;
in a fifth step S₅, the symmetrical or unsymmetrical block copolymer (A₂B₁A₁)_{0.5}I(A₃B₁A₂)_{0.5}, (A₂C₁B₁A₁)_{0.5}I(A₁B₁C₁A₂)_{0.5}, IA₁B₁A₂ or IA₁B₁C₁A₂ obtained in step S₄ is copolymerized with from 80 to 1000 mol of a first epoxide, based on the molar amount of the initiator I, to give a symmetrical or unsymmetrical block copolymer (B₂A₂B₁A₁)_{0.5}I(A₁B₁A₂B₂)_{0.5}, (B₂A₂C₁B₁A₁)_{0.5}I(A₁B₁C₁A₂B₂)_{0.5}, IA₁B₁A₂B₂ or IA₁B₁C₁A₂B₂ and subsequently copolymerized with from 80 to 1000 mol of a second epoxide, based on the molar amount of the initiator I, to give a symmetrical or unsymmetrical block copolymer (C₂B₂A₂B₁A₁)_{0.5}I(A₁B₁A₂B₂C₂)_{0.5}, (C₂B₂A₂C₁B₁A₁)_{0.5}I(A₁B₁C₁A₂B₂C₂)_{0.5}, IA₁B₁A₂B₂C₂ or IA₁B₁C₁A₂B₂C₂, where the first and second epoxide are selected from the group consisting of ethylene oxide (EO), propylene oxide (PO), 1-ethoxyethyl glycidyl ether (EEGE) and glycidol and are different from one another;
the steps S₄ and S₅ are repeated alternately one or more times using an alkyl glycidyl ether of the type (I), (II) or (III) or mixtures thereof selected independently of the preceding steps or with one or two different first and second epoxides or mixtures of a plurality of epoxides which are selected independently of the preceding steps from the group consisting of ethylene oxide (EO), propylene oxide (PO), 1-ethoxyethyl glycidyl ether (EEGE) and glycidol;
in a second step S₂, the initiator I provided in step S₁ is polymerized with from 80 to 1000 mol of an epoxide, based on the molar amount of the initiator I, to give a symmetrical or unsymmetrical oligomer (B₁)_{0.5}I(B₁)_{0.5} or IB₁, where the epoxide is selected from the group consisting of ethylene oxide (EO), propylene oxide (PO), 1-ethoxyethyl glycidyl ether (EEGE) and glycidol;
in a second step S₂, the initiator I provided in step S₁ is copolymerized with a mixture of a total of from 80 to 1000 mol of two, three or four different epoxides, based on the molar amount of the initiator I, to give a symmetrical or unsymmetrical block copolymer (B₁)_{0.5}I(B₁)_{0.5} or IB₁, where the two, three or four epoxides are selected from the group consisting of ethylene oxide (EO), propylene oxide (PO), 1-ethoxyethyl glycidyl ether (EEGE) and glycidol;
in a second step S₂, the initiator I provided in step S₁ is copolymerized with from 80 to 1000 mol of a first epoxide, based on the molar amount of the initiator I, to give a symmetrical or unsymmetrical oligomer (B₁)_{0.5}I(B₁)_{0.5} or IB₁ and subsequently copolymerized with from 80 to 1000 mol of a second epoxide, based on the molar amount of the initiator I, to give a symmetrical or unsymmetrical oligomer (C₁B₁)_{0.5}I(B₁C₁)_{0.5} or IB₁C₁, where the first and second epoxide are selected from the group consisting of ethylene oxide (EO), propylene oxide (PO), 1-ethoxyethyl glycidyl ether (EEGE) and glycidol and are different from one another;
in a third step S₃, the symmetrical or unsymmetrical oligomer (B₁)_{0.5}I(B₁)_{0.5}, (C₁B₁)_{0.5}I(B₁C₁)_{0.5}, IB₁ or IB₁C₁ obtained in step S₂ is copolymerized with from 2 to 40 mol of an alkyl glycidyl ether of the type (I), (II) or (III), a mixture of two or three of the alkyl glycidyl ethers (I), (II), (III) or a mixture of at least one alkyl glycidyl ether (I), (II), (III) with ethylene oxide (EO) and/or 1-ethoxyethyl glycidyl ether (EEGE), based on the molar amount of the initiator I, to give a symmetrical or unsymmetrical block copolymer (A₁B₁)_{0.5}I(B₁A₁)_{0.5}, (A₁C₁B₁)_{0.5}I(B₁C₁A₁)_{0.5}, IB₁A₁ or IB₁C₁A₁;
in a fourth step S₄, the symmetrical or unsymmetrical block copolymer (A₁B₁)_{0.5}I(B₁A₁)_{0.5}, (A₁C₁B₁)_{0.5}I(B₁C₁A₁)_{0.5}, IB₁A₁ or IB₁C₁A₁ obtained in step S₃ is copolymerized with from 80 to 1000 mol of an epoxide, based on the molar amount of the initiator I, to give a symmetrical or unsymmetrical block copolymer (B₂A₁B₁)_{0.5}I(B₁A₁B₂)_{0.5}, (B₂A₁C₁B₁)_{0.5}I(B₁C₁A₁B₂)_{0.5}, IB₁A₁B₂ or IB₁C₁A₁B₂, where the epoxide is selected from the group consisting of ethylene oxide (EO), propylene oxide (PO), 1-ethoxyethyl glycidyl ether (EEGE) and glycidol;
in a fourth step S₄, the symmetrical or unsymmetrical block copolymer (A₁B₁)_{0.5}I(B₁A₁)_{0.5}, (A₁C₁B₁)_{0.5}I(B₁C₁A₁)_{0.5}, IB₁A₁ or IB₁C₁A₁ obtained in step S₃ is copolymerized with a mixture of a total of from 80 to 1000 mol of two, three or four different epoxides, based on the molar amount of the initiator I, to give a symmetrical or unsymmetrical block copolymer (B₂A₁B₁)_{0.5}I(B₁A₁B₂)_{0.5}, (B₂A₁C₁B₁)_{0.5}I(B₁C₁A₁B₂)_{0.5}, IB₁A₁B₂ or IB₁C₁A₁B₂, where the two, three or four epoxides are selected from the group consisting of ethylene oxide (EO), propylene oxide (PO), 1-ethoxyethyl glycidyl ether (EEGE) and glycidol;
in a fourth step S₄, the symmetrical or unsymmetrical block copolymer (A₁B₁)_{0.5}I(B₁A₁)_{0.5}, (A₁C₁B₁)_{0.5}I(B₁C₁A₁)_{0.5}, IB₁A₁ or IB₁C₁A₁ obtained in step S₃ is copolymerized with from 80 to 1000 mol of a first epoxide, based on the molar amount of the initiator I, to give a symmetrical or unsymmetrical block copolymer (B₂A₁B₁)_{0.5}I(B₁A₁B₂)_{0.5}, (B₂A₁C₁B₁)_{0.5}I(B₁C₁A₁B₂)_{0.5}, IB₁A₁B₂ or IB₁C₁A₁B₂ and subsequently copolymerized with from 80 to 1000 mol of a second epoxide, based on the molar amount of the initiator I, to give a symmetrical or unsymmetrical block copolymer (C₂B₂A₁B₁)_{0.5}I(B₁A₁B₂C₂)_{0.5}, (C₂B₂A₁C₁B₁)_{0.5}I(B₁C₁A₁B₂C₂)_{0.5}, IB₁A₁B₂C₂ or IB₁C₁A₁B₂C₂, where the first and second epoxide are selected from the group consisting of ethylene oxide (EO), propylene oxide (PO), 1-ethoxyethyl glycidyl ether (EEGE) and glycidol and are different from one another;
the steps S₃ and S₄ are repeated alternately one or more times using an alkyl glycidyl ether of the type (I), (II) or (III) or a mixture thereof selected independently of the preceding steps or with one or two different first and second epoxides or mixtures of a plurality of epoxides selected independently of the preceding steps from the group consisting of ethylene oxide (EO), propylene oxide (PO), 1-ethoxyethyl glycidyl ether (EEGE) and glycidol;
an aqueous solution of a weak acid is added to the reaction mixture in order to unprotect blocks of polyethoxyethylene glycidyl ether (PEEGE) and hydrolyze them to linear polyglycidol (PG);
an aqueous solution of methanoic acid is added to the reaction mixture in order to unprotect blocks of polyethoxyethylene glycidyl ether (PEEGE) and hydrolyze them to linear polyglycidol (PG);
one or more polymerization steps are carried out by means of anionic ring opening polymerization (ROP);
all polymerization steps are carried out by means of anionic ring opening polymerization (ROP);
all process steps are carried out in a reaction mixture containing one or more deprotonating agents;
all process steps are carried out in a reaction mixture containing one or more deprotonating bases;
all process steps are carried out in a reaction mixture containing one or more deprotonating bases, where the at least one base comprises a counterion such as potassium, lithium and sodium;
all process steps are carried out in a reaction mixture containing one or more deprotonating bases such as potassium tert-butoxide, n-butyllithium and sodium ethoxide;
all process steps are carried out in a reaction mixture containing one or more agents for complexing a counterion;
all process steps are carried out in a reaction mixture containing one or more agents for complexing a counterion, for example potassium, lithium and sodium;
all process steps are carried out in a reaction mixture containing one or more crown ethers for complexing a counterion;
all process steps are carried out in a reaction mixture containing one or more crown ethers for complexing a counterion, for example [18]crown-6, [15]crown-5 or [12]crown-4;
all process steps are carried out in a reaction mixture containing a potassium base and [18]crown-6;
all process steps are carried out in a reaction mixture containing a sodium base and [15]crown-5;
all process steps are carried out in a reaction mixture containing a lithium base and [12]crown-4;
all process steps are carried out in a reaction mixture containing one or more solvents;
all process steps are carried out in a reaction mixture containing one or more solvents such as benzene, methanol, hexane, toluene, tetrahydrofuran, dioxane and dimethyl sulfoxide;
the reaction mixture is homogenized by means of a mechanical method, for example stirring or swirling;
one or more process steps are carried out at a temperature of from 20 to 35° C.;
one or more process steps are carried out at a temperature of from 40 to 60° C., from 50 to 70° C., from 60 to 80° C., from 70 to 90° C., from 80 to 100° C. or from 90 to 110° C.;
one or more polymerization steps are carried out at a temperature of from 40 to 60° C., from 50 to 70° C., from 60 to 80° C., from 70 to 90° C., from 80 to 100° C. or from 90 to 110° C.;
one or more process steps are carried out at a temperature of from −20 to 0° C., from −30 to −10° C., from −40 to −20° C., from −50 to −30° C., from −60 to −40° C., from −70 to −50° C., from −80 to −60° C., from −90 to −70° C. or from −100 to −80° C.;
the addition of epoxides selected from the group consisting of ethylene oxide (EO), propylene oxide (PO), 1-ethoxyethyl glycidyl ether (EEGE) and glycidol is carried out at a temperature of from −20 to 0° C., from −30 to −10° C., from −40 to −20° C., from −50 to −30° C., from −60 to −40° C., from −70 to −50° C., from −80 to −60° C., from −90 to −70° C. or from −100 to −80° C.;
one or more process steps are carried out at a pressure of from 0.9 to 1.1 bar;
one or more process steps are carried out at a pressure of <0.9 bar, <0.5 bar or <0.1 bar; and/or
one or more process steps are carried out at a pressure of from 1.1 to 10 bar, from 5 to 15 bar or from 10 to 30 bar.

Preference is given to carrying out all process steps at a pressure of from 0.9 to 1.1 bar. However, it can be advantageous in individual cases to carry out some of the process steps at increased or reduced pressure.

In general, the polymerization of alkyl glycidyl ethers of the type (I), (II), (III) and of epoxides such as ethylene oxide (EO), propylene oxide (PO), 1-ethoxyethyl glycidyl ether (EEGE), glycidol and mixtures of these epoxides occurs over a period of time which is long compared to the time required for homogenizing the reaction mixture by means of conventional mechanical methods such as stirring or swirling.

In exceptional cases in which the polymerization proceeds rapidly, units of the alkyl glycidyl ether of the type (I), (II) or (III) and/or epoxide monomers such as ethylene oxide (EO), propylene oxide (PO), 1-ethoxyethyl glycidyl ether (EEGE), glycidol or a mixture of these epoxides are added at reduced temperature and the reaction mixture is homogenized by means of stirring or swirling. The temperature is subsequently increased in order to initiate the polymerization.

The invention encompasses block copolymers which are able to be produced by a process comprising one or more of the above-described steps.

A further object of the invention is to provide a block copolymer which is swellable in water or alcohol/water mixtures and has a storage capability for hydrophobic substances.

This object is achieved by a block copolymer having the structure A₁IA₁, [Πᵢ₌₁^{N}AᵢBᵢ]_{0.5}I[Πᵢ₌₁^{N}AᵢBᵢ]_{0.5}, [Πᵢ₌₁^{N}Aᵢ(BᵢCᵢ)]_{0.5}I[┌ᵢ₌₁^{N}Aᵢ(BᵢCᵢ)]_{0.5}, I[Πᵢ₌₁^{N}AᵢBᵢ] or I[Πᵢ₌₁^{N}Aᵢ(BᵢCᵢ)]where N=1, 2, 3, 4, 5, 6, 7, 8, 9 or 10, wherein each of the blocks Aᵢ consists independently of a residual group of an oligomer formed by from 1 to 40 alkyl glycidyl ether units of the type (I), (II) or (III)

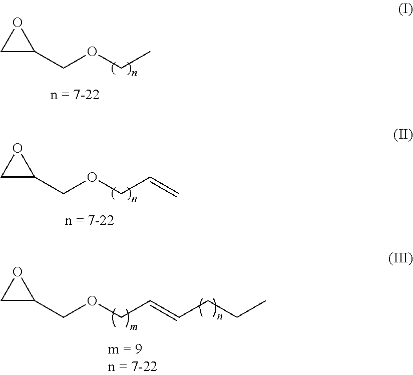

or a residual group of a random cooligomer having from 2 to 40 units of two or three alkyl glycidyl ethers (I), (II), (III) or having from 2 to 40 units of at least one alkyl glycidyl ether (I), (II), (III) and at least one of the epoxides ethylene oxide (EO) and 1-ethoxyethyl glycidyl ether (EEGE);

each of the blocks Bᵢ consists independently of a residual group of a polyether comprising from 80 to 1000 epoxide units, for example polyethylene oxide (PEO), polypropylene oxide (PPO), polyethoxyethylene glycidyl ether (PEEGE), polyglycidol (PG) or a random copolymer of two, three or four different epoxide units such as ethylene oxide (EO), propylene oxide (PO), 1-ethoxyethyl glycidyl ether (EEGE) and/or glycidol;

each of the blocks Cᵢ consists independently of a residual group of a polyether comprising from 80 to 1000 epoxide units, for example polyethylene oxide (PEO), polypropylene oxide (PPO), polyethoxyethylene glycidyl ether (PEEGE) or polyglycidol (PG); and

I is a residual group of an opened alkyl glycidyl ether of the type (I), (II) or (III); or

I is a residual group of a polyether comprising from 80 to 1000 epoxide units, for example polyethylene oxide (PEO), polypropylene oxide (PPO), polyethoxyethylene glycidyl ether (PEEGE), linear or branched polyglycidol (PG, hbPG), monomethyl polyethylene oxide (mPEO), monomethyl propylene oxide (mPPO), monobutyl propylene oxide (mPBO) or a random copolymer of two, three or four different epoxide units such as ethylene oxide (EO), propylene oxide (PO), 1-ethoxyethyl glycidyl ether (EEGE) and/or glycidol;

or

I is a residual group of an alcohol such as methanol, butanol, 2-(benzyloxy)ethanol, pentaerythritol, 1,1,1-trimethylolpropane (TMP), bisphenol A, CH₃(CH₂)ₜOH or OH(CH₂)ₜOH where t=1-21.

The above object is likewise achieved by a block copolymer having the structure A₁IA₁, [Πᵢ₌₁^{N}AᵢBᵢ]_{0.5}I[Πᵢ₌₁^{N}AᵢBᵢ]_{0.5}, [Πᵢ₌₁^{N}Aᵢ(BᵢCᵢ)]_{0.5}I[Πᵢ₌₁^{N}Aᵢ(BᵢCᵢ)]_{0.5}, I[Πᵢ₌₁^{N}AᵢBᵢ] or I[Πᵢ₌₁^{N}Aᵢ(BᵢCᵢ)] where N=1, 2, 3, 4, 5, 6, 7, 8, 9 or 10, wherein each of the blocks Aᵢ consists independently of a residual group of an oligomer formed by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 23, 33, 34, 35, 36, 37, 38, 39 or 40 alkyl glycidyl ether units of the type (I), (II) or (III)

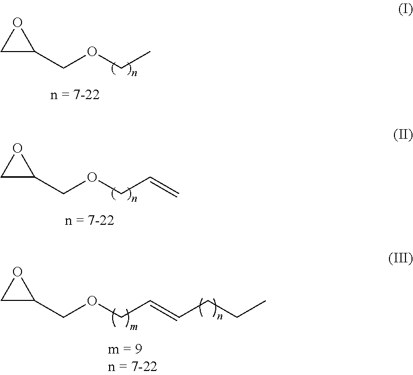

or a residual group of a random cooligomer having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 23, 33, 34, 35, 36, 37, 38, 39 or 40 units of two or three alkyl glycidyl ethers (I), (II), (III) or having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 23, 33, 34, 35, 36, 37, 38, 39 or 40 units of at least one alkyl glycidyl ether (I), (II), (III) and at least one of the epoxides ethylene oxide (EO) and 1-ethoxyethyl glycidyl ether (EEGE);

each of the blocks B, consists independently of a residual group of a polyether comprising from 80 to 1000 epoxide units, for example polyethylene oxide (PEO), polypropylene oxide (PPO), polyethoxyethylene glycidyl ether (PEEGE), polyglycidol (PG) or a random copolymer of two, three or four different epoxide units such as ethylene oxide (EO), propylene oxide (PO), 1-ethoxyethyl glycidyl ether (EEGE) and/or glycidol;

each of the blocks C, consists independently of a residual group of a polyether comprising from 80 to 1000 epoxide units, for example polyethylene oxide (PEO), polypropylene oxide (PPO), polyethoxyethylene glycidyl ether (PEEGE) or polyglycidol (PG); and

I is a residual group of an opened alkyl glycidyl ether of the type (I), (II) or (III); or

I is a residual group of a polyether comprising from 80 to 1000 epoxide units, for example polyethylene oxide (PEO), polypropylene oxide (PPO), polyethoxyethylene glycidyl ether (PEEGE), linear or branched polyglycidol (PG, hbPG), monomethyl polyethylene oxide (mPEO), monomethyl propylene oxide (mPPO), monobutyl propylene oxide (mPBO) or a random copolymer of two, three or four different epoxide units such as ethylene oxide (EO), propylene oxide (PO), 1-ethoxyethyl glycidyl ether (EEGE) and/or glycidol; or

I is a residual group of an alcohol such as methanol, butanol, benzyl alcohol (BnOH), 2-(benzyloxy)ethanol, pentaerythritol, 1,1,1-trimethylolpropane (TMP), bisphenol A, CH₃(CH₂)ₜOH or OH(CH₂)ₜOH where t=1-21.

Advantageous embodiments of the block copolymer of the invention are characterized in that
7≤n≤12, 10≤n≤15, 12≤n≤17, 15≤n≤20 or 17≤n≤22;
the polyether residual groups Bᵢ and Cᵢ are different from one another;
all polyether residual groups Aᵢ are identical;
all polyether residual groups Bᵢ are identical;
all polyether residual groups Cᵢ are identical;
all polyether residual groups Aᵢ are residual groups of random cooligomers of two or three alkyl glycidyl ethers of the type (I), (II) and/or (III);
all polyether residual groups Aᵢ are residual groups of random cooligomers of at least one alkyl glycidyl ether (I), (II), (III) with ethylene oxide (EO) and/or 1-ethoxyethyl glycidyl ether (EEGE);
all polyether residual groups Aᵢ are residual groups of random cooligomers of at least one alkyl glycidyl ether (I), (II), (III) with ethylene oxide (EO) and/or 1-ethoxyethyl glycidyl ether (EEGE), where the proportion of EO and/or EEGE is up to 80% by weight;
all polyether residual groups Bᵢ are radicals of random copolyethers of two, three or four epoxide units such as ethylene oxide (EO), propylene oxide (PO), 1-ethoxyethyl glycidyl ether (EEGE) and/or glycidol;
the block copolymer has a polydispersity M̅_{w}/M̅ₙ≤2;
the block copolymer has a polydispersity M̅_{w}/M̅ₙ≤1.6; preferably M̅_{w}/M̅ₙ≤1.2 and in particular M̅_{w}/M̅ₙ≤1.1;
the block copolymer has a molar mass MW such that 4000 g·mol⁻¹≤MW≤40 000 g·mol⁻¹; and/or
the block copolymer has a molar mass MW such that 4000 g·mol⁻¹≤MW≤20 000 g·mol⁻¹,
15 000 g·mol⁻¹≤MW≤25 000 g·mol⁻¹,
20 000 g·mol⁻¹≤MW≤30 000 g·mol⁻¹,
25 000 g·mol⁻¹≤MW≤35 000 g·mol⁻¹ or
30 000 g·mol⁻¹≤MW≤40 000 g·mol⁻¹.

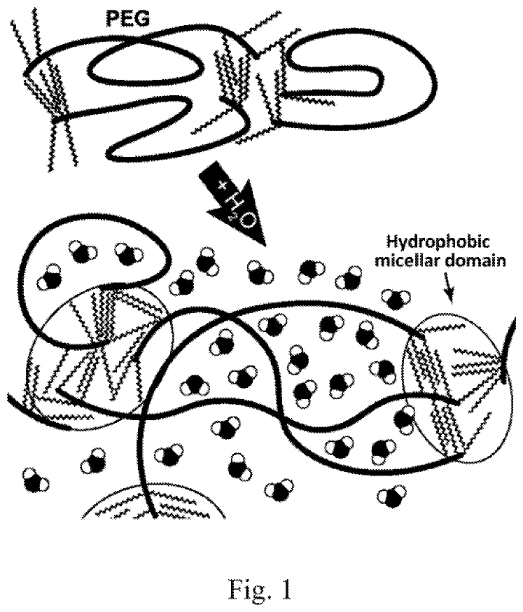

The schematic depiction in FIG. 1 illustrates the mechanisms critical for the functionality of the block copolymers of the invention, namely the swelling by means of water or mixtures of water and alcohol and the formation of micellar hydrophobic domains. The formation of micellar hydrophobic domains in water or aqueous mixtures reduces the free energy and brings about aggregation and mutual alignment of the alkyl segments of the alkyl glycidyl ether blocks to form locally crystalline structures. The micellar hydrophobic and partially crystalline domains bind and store organic active substance molecules and are characterized by a high uptake capability (capacity). When the temperature is increased, the partially crystalline domains melt and the organic active substance molecules bound therein are released.

Owing to their functionality, the block copolymers of the invention are outstandingly suitable for the production of pharmaceutical formulations with controlled release. For this purpose, the block copolymer is dissolved in a water-miscible solvent and mixed with the active substance. Subsequent replacement of the solvent by water or aqueous-alcoholic solutions produces a gel in which the hydrophobic active substance is incorporated into the hydrophobic, micellar domains.

Accordingly, the invention encompasses pharmaceutical retard systems, pharmaceutical administration systems with controlled release and/or pharmaceutical formulations with controlled release, which comprise one or more of the above-described block copolymers.

For the purposes of the present invention, indications of amounts like “from 2 to 40 mol of an alkyl glycidyl ether . . . based on the molar amount of the initiator” and “from 80 to 1000 mol of an epoxide . . . based on the molar amount of the initiator” refer to ratios of from 20 to 40 alkyl glycidyl ether units per initiator molecule and from 80 to 1000 epoxide units per initiator molecule, respectively.

For the purposes of the present invention, Aᵢ is, in each case independently of Aⱼ where j≠i, one of the following residual groups (I′), (II′), (III′)

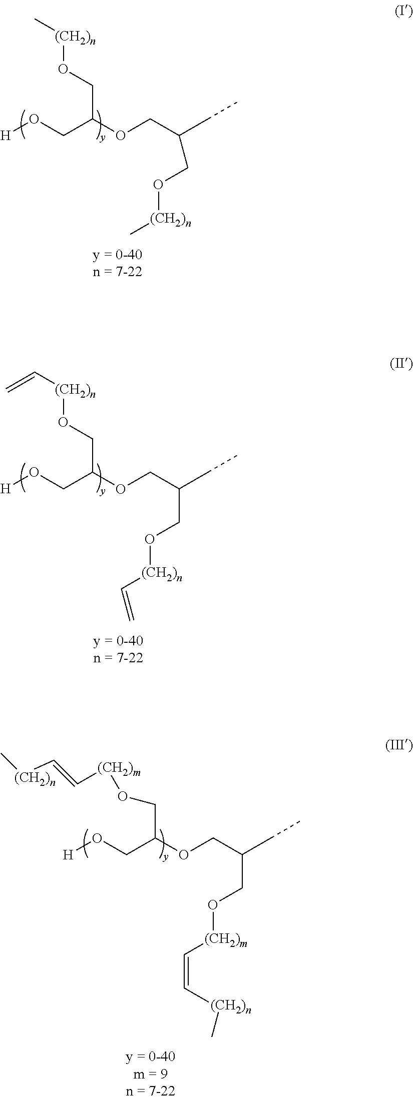

of an oligomer of up to 40 units of an alkyl glycidyl ether of the type (I), (II), (III), a residual group of a random cooligomer of from 2 to 40 units of two or three alkyl glycidyl ethers of the type (I), (II), (III) or a residual group of a random cooligomer of from 2 to 40 units of at least one alkyl glycidyl ether of the type (I), (II), (III) and at least one of the epoxides ethylene oxide (EO) and 1-ethoxyethyl glycidyl ether (EEGE).

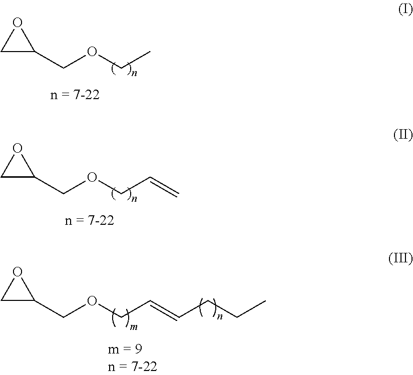

The number of alkyl glycidyl ether units of the type (I), (II), (III) in the oligomers (I′), (II′), (III′) or units in the random cooligomers containing alkyl glycidyl ether units of the type (I), (II), (III) can assume any value in the range from 1 to 40 or from 2 to 40, respectively, i.e. y=0, y=1, y=2, y=3, y=4, y=5, y=6, y=7, y=8, y=9, y=10, y=11, y=12, y=13, y=14, y=15, y=16, y=17, y=18, y=19, y=20, y=21, y=22, y=23, y=24, y=25, y=26, y=27, y=28, y=29, y=30, y=31, y=32, y=33, y=34, y=35, y=36, y=37, y=38, y=39 or y=40.

Accordingly, each of the blocks A, comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 units.

For the purposes of the present invention, Bᵢ and Cᵢ are each, in each case independently of Bⱼ where j≠i or in each case independently of Cⱼ where j≠i, respectively, one of the following residual groups of a polyether comprising from 80 to 1000 epoxide units, for example polyethylene oxide (PEO), polypropylene oxide (PPO), polyethoxyethylene glycidyl ether (PEEGE) or linear polyglycidol (PG),

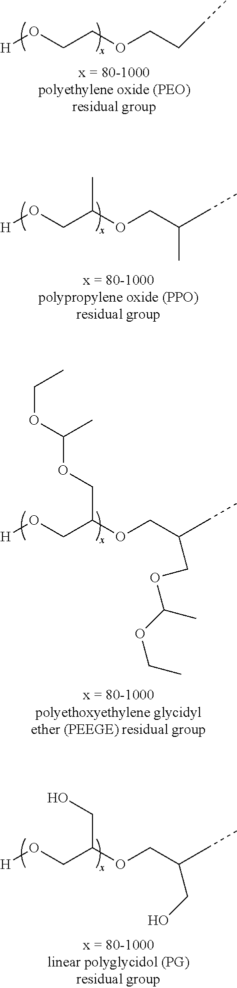

Linear polyglycidol (PG) can be produced by polymerization of 1-ethoxyethyl glycidyl ether and subsequent unprotection and hydrolysis by means of a weak acid. Apart from linear polyglycidol (PG), it is possible for the blocks Bᵢ and Cᵢ also to consist of branched polyglycidol (hbPG). Branched polyglycidol (hbPG) is obtained by polymerization of the epoxide glycidol.

Furthermore, for the purposes of the present invention, Bᵢ is also a residual group of a random copolymer comprising from 80 to 1000 epoxide units or cooligomer of two, three or four different epoxide units selected from the group consisting of ethylene oxide (EO), propylene oxide (PO), 1-ethoxyethyl glycidyl ether (EEGE) and glycidol.

The structure of block copolymers of the invention is described by the formulae

A₁IA₁,

[Πᵢ₌₁^{N}AᵢBᵢ]_{0.5}I[Πᵢ₌₁^{N}AᵢBᵢ]_{0.5},

[Πᵢ₌₁^{N}Aᵢ(BᵢCᵢ)]_{0.5}I[Πᵢ₌₁^{N}Aᵢ(BᵢCᵢ)]_{0.5},

I[Πᵢ₌₁^{N}AᵢBᵢ] and

I[Πᵢ₌₁^{N}Aᵢ(BᵢCᵢ)]

where N=1, 2, 3, 4, 5, 6, 7, 8, 9 or 10,

which represent the structural quantity S, where

S={A₁IA₁, (B₁A₁)_{0.5}I(A₁B₁)_{0.5}, IA₁B₁, (C₁B₁A₁)_{0.5}I(A₁B₁C₁)_{0.5}, IA₁B₁C₁, (A₂B₁A₁)_{0.5}I(A₁B₁A₂)_{0.5}, IA₁B₁A₂, (A₂C₁B₁A₁)_{0.5}I(A₁B₁C₁A₂)_{0.5}, IA₁B₁C₁A₂, (B₂A₂B₁A₁)_{0.5}I(A₁B₁A₂B₂)_{0.5}, IA₁B₁A₂B₂, (B₂A₂C₁B₁A₁)_{0.5}I(A₁B₁C₁A₂B₂)_{0.5}, IA₁B₁C₁A₂B₂, (C₂B₂A₂B₁A₁)_{0.5}I(A₁B₁A₂B₂C₂)_{0.5}, IA₁B₁A₂B₂C₂, (C₂B₂A₂C₁B₁A₁)_{0.5}I(A₁B₁C₁A₂B₂C₂)_{0.5}, IA₁B₁C₁A₂B₂C₂, . . . }.

As explained above in connection with the process of the invention, the above structural formulae encompass, depending on the sequence of the process steps, structures in which the sequence of the blocks Aᵢ has been exchanged with the blocks Bᵢ and (BᵢCᵢ).

According to the invention, the structural formulae [Πᵢ₌₁^{N}AᵢBᵢ]_{0.5}I[Πᵢ₌₁^{N}AᵢBᵢ]_{0.5} and [Πᵢ₌₁^{N}Aᵢ(BᵢCᵢ)]_{0.5}I[Πᵢ₌₁^{N}Aᵢ(BᵢCᵢ)]_{0.5} designate symmetrical and pseudo-symmetrical block copolymers in which the blocks Aᵢ, Bᵢ, Cᵢ in the in each case two segments [Πᵢ₌₁^{N}AᵢBᵢ]_{0.5} and [Πᵢ₌₁^{N}Aᵢ(BᵢCᵢ)]_{0.5} conjugated with the initiator I consist of the same number of monomers or numbers of monomers which differ by 1. Where the respective number of monomers in the blocks Aᵢ, Bᵢ, Cᵢ in the two segments [Πᵢ₌₁^{N}AᵢBᵢ]_{0.5} and [Πᵢ₌₁^{N}Aᵢ(BᵢCᵢ)]_{0.5} conjugated with the initiator I are equal or differ by 1 depends on whether the number of mol used for polymerization of the blocks Aᵢ, Bᵢ, Cᵢ (from 1 to 40 for Aᵢ and from 80 to 1000 for Bᵢ and Cᵢ, in each case based on the initiator) is even or odd.

For the purposes of the invention, the terms “symmetrical” and “unsymmetrical” do not refer to chemical structures having point or mirror symmetry. Rather, the terms “symmetrical” and “unsymmetrical” relate to the polymerization sequence which forms the segments [Πᵢ₌₁^{N}AᵢBᵢ]_{0.5}, [Πᵢ₌₁^{N}Aᵢ(BᵢCᵢ)]_{0.5} and [Πᵢ₌₁^{N}AᵢBᵢ], [Πᵢ₌₁^{N}Aᵢ(BᵢCᵢ)] proceeding from the initiator I.

The epoxides used in the process of the invention and their usual oligomers are shown below:

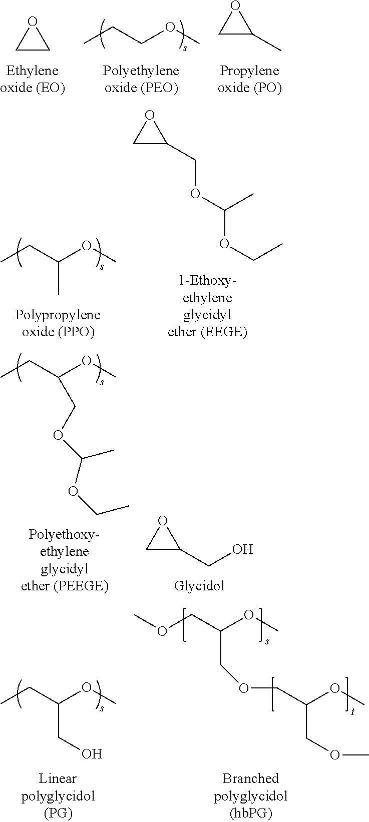

### Measurement Methods

All chemicals and solvents were, unless listed separately, procured from commercial suppliers (Acros, Sigma-Aldrich, Fisher Scientific, Fluka, Riedel-de-Haën, Roth) and used without further purification. Deuterated solvents were procured from Deutero GmbH (Kastellaun, Germany). All experiments were, unless indicated otherwise, carried out at room temperature (20-25° C.), atmospheric pressure (985-1010 hPa) and typical atmospheric humidity (40-100% rH). (Source: measurement station of Institut für Physik der Atmosphare, Johannes Gutenberg University, Mainz).

### NMR Spectroscopy

¹H- and ¹³C-NMR spectra were recorded on an Avance III HD 300 (300 MHz, 5 mm BBFO head with z gradient and ATM from Bruker at a frequency of 300 MHz (¹H) or 75 MHz (¹³C). Spectra at 400 MHz (¹H) were recorded on an Avance II 400 (400 MHz, 5 mm BBFO head with z gradient and ATM) from Bruker. The chemical shifts are reported in ppm and are relative to the proton signal of the deuterated solvent.

### Gel Permeation Chromatography (GPC)

The GPC measurements were carried out in accordance with DIN 55672-3 2016-01 using dimethylformamide (DMF) admixed with 1 g/l of lithium bromide as eluent on an Agilent 1100 series instrument with an HEMA 300/100/40 column from MZ-Analysetechnik. Detection of the signals was carried out by means of an RI detector (Agilent G1362A) and UV (254 nm) detector (Agilent G1314A). Recording of the GPC rate and curves was carried out using primarily the signal of the RI detector and optionally the signal of the UV detector. The measurements were carried out at 50° C. and a flow rate of 1.0 ml/min. Calibration was carried out using polyethylene glycol standards 200, 1000, 2000, 6000, 20 000 and 40 000 and polystyrene standards from Polymer Standard Service.

When using the solvent THF, this is introduced by means of a Waters 717 plus injector into a column of the type MZ-Gel SD plus e5/e3/100. An RI detector model Agilent 2160 Infinity is used for the measurement. The eluent is degassed by means of a degasser model ERC-3315a and a flow rate of 1.0 ml/min is set using a Spectra Series P1000 pump. The measurement is carried out at a temperature of 25° C. A poly(ethylene glycol) standard from Polymer Standard Service was used for calibration. In addition, a toluene standard was used. The injection volume is 100 μl. The elution graphs are evaluated by means of the software PSS WinGPC Unity.

In the context of the present invention, the following abbreviations are used:

PE . . . petroleum ether

EA . . . ethyl acetate

DCM . . . dichloromethane

CSA . . . DL-camphor-10-sulfonic acid

DMP . . . 2,2-dimethoxypropane

THF . . . tetrahydrofuran

DMF . . . dimethylformamide

eq . . . equivalents

EO . . . ethylene oxide

PO . . . propylene oxide

EEGE . . . 1-ethoxyethyl glycidyl ether

D . . . polydispersity

The invention will be illustrated below with the aid of examples.

### PHDGE₆-b-PEG₁₃₆-b-PHDGE₆

In a 50 ml Schlenk flask with septum, 1 g (0.20 mmol, 1 eq.) of polyethylene glycol (Mw=6000 g/mol), 30 mg (0.26 mmol, 1.6 eq.) of potassium tert-butoxide and 88 mg (0.33 mmol, 2 eq.) of [18]crown-6 crown ether were dissolved in 10 ml of benzene and 1.5 ml of methanol. A gentle, static vacuum was applied to the flask so that the benzene began to boil and the mixture was subsequently stirred at 60° C. for 30 minutes. The reaction mixture was subsequently dried overnight at 60° C. in a high vacuum. After drying was complete, the reaction flask was flooded with argon, and 0.68 ml (2.00 mmol, 12 eq.) of hexadecyl glycidyl ether (HDGE) was added through the septum by means of a syringe. The reaction mixture was subsequently stirred at 80° C. for 24 hours under an argon atmosphere.

After the reaction was complete, the reaction mixture was dissolved in 3 ml of dichloromethane at a temperature of 25° C. and subsequently added dropwise to about 40 ml of diethyl ether. After allowing to stand at room temperature for 2 hours, the precipitated polymer was centrifuged and decanted. The remaining solvent was removed at 40° C. under reduced pressure.

M_{w}(¹H-NMR)=9 580 g/mol Mₙ(GPC)=8 300 g/mol M_{w}/Mₙ(GPC*)=1.06

### PDDGE₇-b-PEG₂₂₇-b-PDDGE₇

In a 50 ml Schlenk flask with septum, 1 g (0.10 mmol, 1 eq.) of polyethylene glycol (Mw=10 000 g/mol), 18 mg (0.16 mmol, 1.6 eq.) of potassium tert-butoxide and 88 mg (0.20 mmol, 2 eq.) of [18]crown-6 crown ether were dissolved in 10 ml of benzene and 1.5 ml of methanol. A gentle, static vacuum was applied to the flask so that the benzene began to boil and the mixture was subsequently stirred at 60° C. for 30 minutes. The reaction mixture was subsequently dried overnight at 60° C. in a high vacuum. After drying was complete, the reaction flask was flooded with argon, and 0.39 ml (1.4 mmol, 14 eq.) of dodecyl glycidyl ether (DDGE) was added through the septum by means of a syringe. The reaction mixture was subsequently stirred at 80° C. for 24 hours under an argon atmosphere.

After the reaction was complete, the reaction mixture was dissolved in 3 ml of dichloromethane at a temperature of 25° C. and subsequently added dropwise to about 40 ml of diethyl ether. After allowing to stand for 2 hours at room temperature, the precipitated polymer was centrifuged and decanted. The remaining solvent was removed at 40° C. under reduced pressure.

M_{w}(¹H-NMR)=13 400 g/mol Mₙ(GPC)=13 000 g/mol M_{w}/Mₙ(GPC*)=1.08

### PDDGE₇-b-PEG₄₅₄-b-EDDGE₇

In a 50 ml Schlenk flask with septum, 1 g (0.05 mmol, 1 eq.) of polyethylene glycol (Mw=20 000 g/mol), 9 mg (0.08 mmol, 1.6 eq.) of potassium tert-butoxide and 26 mg (0.10 mmol, 2 eq.) of [18]crown-6 crown ether were dissolved in 10 ml of benzene and 1.5 ml of methanol. A gentle, static vacuum was applied to the flask so that the benzene began to boil and the mixture was subsequently stirred at 60° C. for 30 minutes. The reaction mixture was subsequently dried overnight at 60° C. in a high vacuum. After drying was complete, the reaction flask was flooded with argon, and 0.19 ml (0.7 mmol, 14 eq.) of dodecyl glycidyl ether (DDGE) was added through the septum by means of a syringe. The reaction mixture was subsequently stirred at 80° C. for 24 hours under an argon atmosphere.

After the reaction was complete, the reaction mixture was dissolved in 3 ml of dichloromethane at a temperature of 25° C. and subsequently added dropwise to about 40 ml of diethyl ether. After allowing to stand for 2 hours at room temperature, the precipitated polymer was centrifuged and decanted. The remaining solvent was removed at 40° C. under reduced pressure.

M_{w}(¹H-NMR)=23 900 g/mol Mₙ(GPC)=20 800 g/mol M_{w}/Mₙ(GPC*)=1.14

### PHDGE₁₄-b-PEG₄₅₄-b-PHDGE₁₄

In a 25 ml Schlenk flask with septum, 2 g (0.1 mmol, 1 eq.) of polyethylene glycol (Mw=20 000 g/mol), 18 mg (0.16 mmol, 1.6 eq.) of potassium tert-butoxide and 53 mg (0.20 mmol, 2 eq.) of [18]crown-6 crown ether were dissolved in 10 ml of benzene and 1.5 ml of methanol. A gentle, static vacuum was applied to the flask so that the benzene began to boil and the mixture was subsequently stirred at 60° C. for 30 minutes. The reaction mixture was subsequently dried overnight at 60° C. in a high vacuum. After drying was complete, the reaction flask was flooded with argon, and 0.95 ml (0.84 mmol, 28 eq.) of hexadecyl glycidyl ether (HDGE) was added through the septum by means of a syringe. The reaction mixture was subsequently stirred at 80° C. for 24 hours under an argon atmosphere.

After the reaction was complete, the reaction mixture was dissolved in 5 ml of dichloromethane at a temperature of 25° C. and subsequently added dropwise to about 40 ml of diethyl ether. After allowing to stand for 2 hours at room temperature, the precipitated polymer was centrifuged and decanted. The remaining solvent was removed at 40° C. under reduced pressure.

M_{w}(¹H-NMR)=28 300 g/mol Mₙ(GPC)=22 000 g/mol M_{w}/Mₙ(GPC*)=1.28

### Setting of the Melting Point by Copolymerization of HDGE and DDGE in a Prescribed Ratio

### BnO-PHDGE₉-co-PDDGE₃

In a 25 ml Schlenk flask with septum, 30 mg (0.2 mmol, 1 eq.) of benzyloxyethanol (BnO), 18 mg (0.018 mmol, 0.8 eq.) of potassium tert-butoxide and 104 mg (0.4 mmol, 2 eq.) of [18]crown-6 crown ether were dissolved in 5 ml of benzene and 1 ml of methanol. A gentle, static vacuum was applied to the flask so that the benzene began to boil and the mixture was subsequently stirred at 60° C. for 30 minutes. The reaction mixture was subsequently dried overnight at 40° C. in a high vacuum. After drying was complete, the reaction flask was flooded with argon, and 0.765 ml of a mixture of 530 mg (1.8 mmol, 9 eq.) of hexadecyl glycidyl ether (HDGE) and 143 mg (0.6 mmol, 3 eq.) of dodecyl glycidyl ether (DDGE) was added through the septum by means of a syringe. The reaction mixture was subsequently stirred at 80° C. for 24 hours under an argon atmosphere.

After the reaction was complete, the reaction mixture was dissolved in 3 ml of dichloromethane at a temperature of 25° C. and subsequently added dropwise to about 40 ml of methanol. The mixture was subsequently stored at −20° C. for 12 hours. The precipitated polymer was centrifuged and decanted. The remaining solvent was removed at 40° C. under reduced pressure.

M_{w}(¹H-NMR)=3410 g/mol Mₙ(GPC)=3000 g/mol M_{w}/Mₙ(GPC**)=1.09

### BnO-PDDGE₁₈-b-PEEGE₂₈

In a 50 ml Schlenk flask with septum, 0.1 g (0.65 mmol, 1 eq.) of benzyloxyethanol (BnO), 66 mg (0.59 mmol, 0.9 eq.) of potassium tert-butoxide and 521 mg (1.97 mmol, 3 eq.) of [18]crown-6 crown ether were dissolved in 10 ml of benzene and 1.5 ml of methanol. A gentle, static vacuum was applied to the flask so that the benzene began to boil and the mixture was subsequently stirred at 60° C. for 30 minutes. The reaction mixture was dried overnight at 60° C. in a high vacuum. After drying was complete, the reaction flask was flooded with argon, and 3.25 ml (11.82 mmol, 18 eq.) of dodecyl glycidyl ether (DDGE) was added through the septum by means of a syringe. The reaction mixture was subsequently stirred at 80° C. for 24 hours under an argon atmosphere. After the reaction was complete, 3.12 ml (21.4 mmol, 28 eq.) of ethoxyethyl glycidyl ether (EEGE) were added and the mixture was stirred at 80° C. for 24 hours under an argon atmosphere. After the reaction was complete, the reaction mixture was dissolved in 3 ml of dichloromethane at a temperature of 25° C. and subsequently added dropwise to about 40 ml of methanol. After allowing to stand at −20° C. for 5 hours, the precipitated polymer was centrifuged and decanted. The remaining solvent was removed at 40° C. under reduced pressure.

* Eluent: DMF, calibration: PEG

** Eluent: THF, calibration: PEG

### Uptake Efficiency

100 mg of the ABA triblock copolymer PDDGE₅-b-PEG₂₂₇-b-PDDGE₅ (hereinafter referred to as PV119) were dissolved in 0.5 ml of a Nile red/THF solution having a concentration c=1.0 g/l, introduced into a dialysis tube composed of regenerated cellulose from Spectrum Laboratories, Biotech, of the type CE Tubing, MWCO: 100-500 D having a flat width of 31 mm, diameter of 20 mm, specific volume of 3.1 ml/cm and length of 10 m and dialyzed for 2 days (2 d) against 1 l of deionized water at a temperature of 30° C. The dialysis water was replaced once by deionized water over the period of 2 d.

For comparison, 100 mg of PEG₂₂₇ (hereinafter referred to as PEG10k) were dialyzed in Nile red/THF solution (c=0.1 g/l) under the same experimental conditions in deionized water in an identical dialysis tube.

After dialysis for two days, the swollen, violet-colored hydrogel of the triblock copolymer PDDGE₅-b-PEG₂₂₇-b-PDDGE₅ was taken from the dialysis tube. 33 mg of gel were dissolved in 3 ml of dichloromethane and subsequently analyzed by means of HPLC.

In an analogous way, 122 mg of the colorless PEG₂₂₇ solution which had been dialyzed for two days were dissolved in 3 ml of dichloromethane and analyzed by means of HPLC.

The HPLC measurements were carried out using a 1260 Infinity System from Agilent Technologies in semipreparative configuration with 1260 QuatPump, 1260 ALS Autosampler, 1260 VWD UV-Vis detector with variable wavelength setting and Softa 1300 evaporative light scattering detector (ELSD). The UV detector was set to a wavelength of 254 nm and the column oven was set to a temperature of 50° C. A silica column from MZ Analysentechnik model PerfectSil having dimensions of 250 mm×4.6 mm, 300 Si 5 μm, was used for the analysis. A mixture of n-hexane (inlet C) and chloroform (inlet B) was used as mobile phase.

An HPLC calibration line was recorded by means of a dilution series of Nile red in dichloromethane. The concentration of the dissolved hydrogel sample was determined by linear regression. The content of Nile red was subsequently calculated on the basis of the total weight of the gel. The measurement results are shown below.

TABLE 1
Calibration values for Nile red in dichloromethane

| | UV signal after base |
|---|---|
| Concentration | line correction |
| 3.91 × 10⁻³ | 0.1794 |
| 1.95 × 10⁻³ | 0.0915 |
| 9.77 × 10⁻⁴ | 0.0480 |
| 4.88 × 10⁻⁴ | 0.0259 |
| 2.44 × 10⁻⁴ | 0.0145 |
| 1.22 × 10⁻⁴ | 0.009 |
| 6.10 × 10⁻⁵ | 0.0066 |

A concentration of the solution of PV119/Nile red in dichloromethane of c=1.9×10⁻⁴ g/l was found.

In an analogous way, a concentration of the solution of PEG10k/Nile red in dichloromethane of c=8.8×10⁻⁶ g/l was found.

Based on these figures, an amount of 1.8×10⁻³ mg of Nile red in 33 mg of PV119 and correspondingly 0.0277 mg in 512 mg of PV119 was determined.

A value of

0.0277 mg/(0.1 mg/l×0.5 ml)=0.554 (55.4%)

is found for the ratio of the amount of Nile red in the dialyzed PV119/Nile red gel to the amount of Nile red initially added.

### Triblock Copolymers

In a manner analogous to the above examples of synthesis for PHDGE₆-b-PEG₁₃₆-b-PHDGE₆, PDDGE₇-b-PEG₂₂₇-b-PDDGE₇, PDDGE₇-b-PEG₄₅₄-b-PDDGE₇ and PHDGE₁₄-b-PEG₄₅₄-b-PHDGE₁₄, further triblock copolymers shown in Table 2 were produced and characterized.

TABLE 2

| | Tₘ^{a} | Mₙ^{b} | Mₙ^{c} | Mₙ^{d} | Ð^{e} |
|---|---|---|---|---|---|
| Triblock copolymer | [° C.] | [g · mol⁻¹] | [g · mol⁻¹] | [g · mol⁻¹] | — |
| PDDGE₃-b-PEG₁₃₆- | 0/53 | 7436 | 7400 | 10 600 | 1.17 |
| b-PDDGE₃ | | | | | |
| PDDGE₅-b-PEG₁₃₆- | 8/52 | 8888 | 8400 | 13 000 | 1.13 |
| b-PDDGE₅ | | | | | |
| PDDGE₅-b-PEG₂₂₇- | 2/57 | 12 892 | 12 400 | 20 200 | 1.12 |
| b-PDDGE₅ | | | | | |
| PDDGE₈-b-PEG₂₂₇- | 10/56 | 13 860 | 13 800 | 24 600 | 1.14 |
| b-PDDGE₈ | | | | | |
| PDDGE₆-b-PEG₄₅₄- | 0/61 | 22 880 | 22 900 | 27 000 | 1.31 |
| b-PDDGE₆ | | | | | |
| PDDGE₁₂-b-PEG₄₅₄- | 11/61 | 26 752 | 25 800 | 30 000 | 1.34 |
| b-PDDGE₁₂ | | | | | |
| PHDGE₃-b-PEG₁₃₆- | 33/52 | 7772 | 7800 | 12 000 | 1.20 |
| b-PHDGE₃ | | | | | |
| PHDGE₅-b-PEG₁₃₆- | 38/50 | 9560 | 9000 | 17 000 | 1.19 |
| b-PHDGE₅ | | | | | |
| PHDGE₅-b-PEG₂₂₇- | 37/52 | 13 564 | 13 600 | 23 000 | 1.14 |
| b-PHDGE₅ | | | | | |
| PHDGE₉-b-PEG₂₂₇- | 40/54 | 15 948 | 15 400 | 28 000 | 1.14 |
| b-PHDGE₉ | | | | | |
| PHDGE₅-b-PEG₄₅₄- | 34/62 | 23 552 | 23 000 | 32 000 | 1.20 |
| b-PHDGE₅ | | | | | |
| PHDGE₁₄-b-PEG₄₅₄- | 41/57 | 28 320 | 28 300 | 43 000 | 1.22 |
| b-PHDGE₁₄ | | | | | |

^{a}first and second melting point for alkyl glycidyl ether and polyethylene glycol;
^{b}calculated molar mass;
^{c}molar mass measured by ¹H NMR (300 MHz, CDCl₃);
^{d}molar mass measured by SEC (eluent THF, calibrated with PEG);
^{e}Ð ≡ polydispersity.

1. A process for producing a block copolymer comprising copolymerizing one or more alkyl glycidyl ethers of the type (I), (II), or (III) 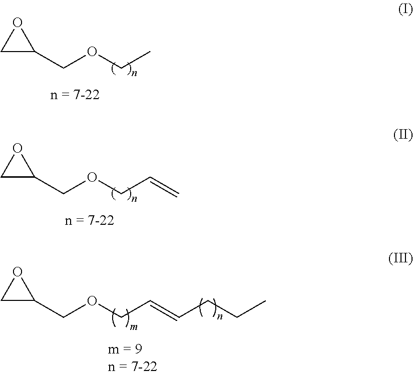
with one or more epoxides selected from the group consisting of ethylene oxide (EO), propylene oxide (PO), 1-ethoxyethyl glycidyl ether (EEGE), glycidol and/or mixtures of two, three or four different epoxides from among these to form blocks composed of polyethylene oxide (PEO), polypropylene oxide (PPO), polyethoxyethylene glycidyl ether (PEEGE), linear or branched polyglycidol (PG, hbPG) and/or random copolymers of the above epoxides; or
with one or more polyethers selected from the group consisting of polyethylene oxide (PEO), polypropylene oxide (PPO), polyethoxyethylene glycidyl ether (PEEGE), linear or branched polyglycidol (PG, hbPG), monomethyl polyethylene oxide (mPEO), monomethyl propylene oxide (mPPO), monobutyl propylene oxide (mPBO) or a random copolymer of two, three or four different epoxide units.

2. The process as claimed in claim 1, wherein said process further comprises providing, in a first step S₁, a reaction mixture with an initiator I selected from the group consisting of
a deprotonated residual group of an opened alkyl glycidyl ether of the type (I), (II) or (III);
a deprotonated residual group of a polyether such as polyethylene oxide (PEO), polypropylene oxide (PPO), polyethoxyethylene glycidyl ether (PEEGE), linear or branched polyglycidol (PG, hbPG), monomethyl polyethylene oxide (mPEO), monomethyl propylene oxide (mPPO), monobutyl propylene oxide (mPBO) or a random copolymer of two, three or four different epoxide units selected from ethylene oxide (EO), propylene oxide (PO), 1-ethoxyethyl glycidyl ether (EEGE) and/or glycidol; and
a deprotonated residual group of an alcohol.

3. The process as claimed in claim 2 further comprising polymerizing, in a second step S₂, the initiator I provided in step S₁ with from 2 to 40 mol of an alkyl glycidyl ether of (I), (II) or (III), a mixture of two or three alkyl glycidyl ethers of (I), (II), (III) or a mixture of at least one alkyl glycidyl ether (I), (II), (III) with ethylene oxide (EO) and/or 1-ethoxyethyl glycidyl ether (EEGE), based on the molar amount of the initiator I, to give a symmetrical or unsymmetrical oligomer (A₁)_{0.5}I(A₁)_{0.5} or IA₁.

4. The process as claimed in claim 3 further comprising copolymerizing, in a third step S₃, the symmetrical or unsymmetrical oligomer (A₁)_{0.5}I(A₁)_{0.5} or IA₁ obtained in step S₂ with from 80 to 1000 mol of an epoxide, based on the molar amount of the initiator I, to give a symmetrical or unsymmetrical block copolymer (B₁A₁)_{0.5}I(A₁B₁)_{0.5} or IA₁B₁, where the epoxide is selected from the group consisting of ethylene oxide (EO), propylene oxide (PO), 1-ethoxyethyl glycidyl ether (EEGE) and glycidol; or,
copolymerizing, in a third step S₃, the symmetrical or unsymmetrical oligomer (A₁)_{0.5}I(A₁)_{0.5} or IA₁ obtained in step S₂ with a mixture of a total of from 80 to 1000 mol of two, three or four different epoxides, based on the molar amount of the initiator I, to give a symmetrical or unsymmetrical block copolymer (B₁A₁)_{0.5}I(A₁B₁)_{0.5} or IA₁B₁, where the two, three or four epoxides are selected from the group consisting of ethylene oxide (EO), propylene oxide (PO), 1-ethoxyethyl glycidyl ether (EEGE) and glycidol; or,
copolymerizing, in a third step S₃, the symmetrical or unsymmetrical oligomer (A₁)_{0.5}I(A₁)_{0.5} or IA₁ obtained in step S₂ with from 80 to 1000 mol of a first epoxide, based on the molar amount of the initiator I, to give a symmetrical or unsymmetrical block copolymer (B₁A₁)_{0.5}I(A₁B₁)_{0.5} or IA₁B₁ and subsequently polymerizing the block copolymer with from 80 to 1000 mol of a second epoxide, based on the molar amount of the initiator I, to give a symmetrical or unsymmetrical block copolymer (C₁B₁A₁)_{0.5}I(A₁B₁C₁)_{0.5} or IA₁B₁C₁, where the first and second epoxide are selected from the group consisting of ethylene oxide (EO), propylene oxide (PO), 1-ethoxyethyl glycidyl ether (EEGE) and glycidol and said first and second epoxide are different from one another.

5. The process as claimed in claim 2, further comprising copolymerizing, in a second step S₂, the initiator I provided in step with from 80 to 1000 mol of an epoxide, based on the molar amount of the initiator I, to give a symmetrical or unsymmetrical oligomer (B₁)_{0.5}I(B₁)_{0.5} or IB₁, where the epoxide is selected from the group consisting of ethylene oxide (EO), propylene oxide (PO), 1-ethoxyethyl glycidyl ether (EEGE) and glycidol; or,
copolymerizing, in a second step S₂, the initiator I provided in step S₁ with a mixture of a total of from 80 to 1000 mol of two, three or four different epoxides, based on the molar amount of the initiator I, to give a symmetrical or unsymmetrical block copolymer (B₁)_{0.5}I(B₁)_{0.5} or IB₁, where the two, three or four epoxides are selected from the group consisting of ethylene oxide (EO), propylene oxide (PO), 1-ethoxyethyl glycidyl ether (EEGE) and glycidol, or,
copolymerizing, in a second step S₂, the initiator I provided in step S₁ with from 80 to 1000 mol of a first epoxide, based on the molar amount of the initiator I, to give a symmetrical or unsymmetrical oligomer (B₁)_{0.5}I(B₁)_{0.5} or IB₁ and subsequently polymerizing the oligomer with from 80 to 1000 mol of a second epoxide, based on the molar amount of the initiator I, to give a symmetrical or unsymmetrical oligomer (C₁B₁)_{0.5}I(B₁C₁)_{0.5} or IB₁C₁, where the first and second epoxide are selected from the group consisting of ethylene oxide (EO), propylene oxide (PO), 1-ethoxyethyl glycidyl ether (EEGE) and glycidol and said first and second epoxide are different from one another.

6. The process as claimed in claim 5, further comprising copolymerizing, in a third step S₃, the symmetrical or unsymmetrical oligomer (B₁)_{0.5}I(B₁)_{0.5}, (C₁B₁)_{0.5}I(B₁C₁)_{0.5}, IB₁ or IB₁C₁ obtained in step S₂ with from 2 to 40 mol of an alkyl glycidyl ether of the type (I), (II) or (III), a mixture of two or three alkyl glycidyl ethers of the type (I), (II), (III) or a mixture of at least one alkyl glycidyl ether (I), (II), (III) with ethylene oxide (EO) and/or 1-ethoxyethyl glycidyl ether (EEGE), based on the molar amount of the initiator I, to give a symmetrical or unsymmetrical block copolymer (A₁B₁)_{0.5}I(B₁A₁)_{0.5}, (A₁C₁B₁)_{0.5}I(B₁C₁A₁)_{0.5}, IB₁A₁ or IB₁C₁A_{1.}

7. The process as claimed in claim 6 further comprising repeating the steps S₂ and S₃ alternately one or more times using an alkyl glycidyl ether of the type (I), (II) or (III), a mixture of two or three alkyl glycidyl ethers of the type (I), (II), (III) or a mixture of at least one alkyl glycidyl ether (I), (II), (III) with ethylene oxide (EO) and/or 1-ethoxyethyl glycidyl ether (EEGE) or using one or two different first and second epoxides or mixtures of a plurality of epoxides which are selected independently of the preceding steps from the group consisting of ethylene oxide (EO), propylene oxide (PO), 1-ethoxyethyl glycidyl ether (EEGE) and glycidol.

8. The process as claimed in claim 1, wherein all process steps are carried out in a reaction mixture containing one or more deprotonated bases, where the at least one base comprises a counterion.

9. The process as claimed in claim 8, wherein all process steps are carried out in a reaction mixture containing one or more crown ethers for complexing a counterion.

10. A block copolymer which produced by the process as claimed in claim 1.

11. A block copolymer having the structure
A₁IA₁,
[Πᵢ₌₁^{N}AᵢBᵢ]_{0.5}I[Πᵢ₌₁^{N}AᵢBᵢ]_{0.5},
[Πᵢ₌₁^{N}Aᵢ(BᵢCᵢ)]_{0.5}I[Πᵢ₌₁^{N}Aᵢ(BᵢCᵢ)]_{0.5},
I[Πᵢ₌₁^{N}AᵢBᵢ] and
I[Πᵢ₌₁^{N}Aᵢ(BᵢCᵢ)]
where N=1, 2, 3, 4, 5, 6, 7, 8, 9 or 10,
each of the blocks Aᵢ consists independently of a residual group of an oligomer formed by from 1 to 40 alkyl glycidyl ether units (I), (II) or (III) 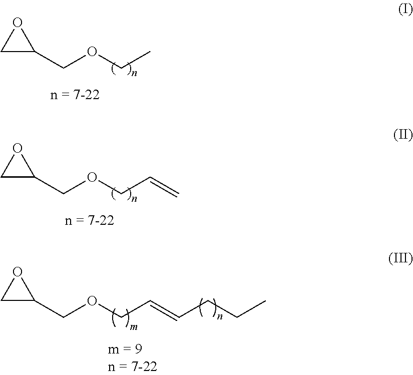
or a residual group of a random cooligomer having from 2 to 40 units of two or three alkyl glycidyl ethers (I), (II), (III) or having from 2 to 40 units of at least one alkyl glycidyl ether (I), (II), (III) and at least one of the epoxides ethylene oxide (EO) and 1-ethoxyethyl glycidyl ether (EEGE);
each of the blocks Bᵢ consists independently of a residual group of a polyether comprising from 80 to 1000 epoxide units, or a random copolymer of two, three or four different epoxide units;
each of the blocks Cᵢ consists independently of a residual group of a polyether comprising from 80 to 1000 epoxide units; and
I is a residual group of an alkyl glycidyl ether of the type (I), (II) or (III); or I is a residual group of a polyether comprising from 80 to 1000 epoxide units, or a random copolymer of two, three or four different epoxide units; or I is a residual group of an alcohol.

12. The block copolymer as claimed in claim 11, wherein the block copolymer has a polydispersity
M̅_{w}/M̅ₙ≤2, M̅_{w}/M̅ₙ≤1.6, M̅_{w}/M̅ₙ≤1.2 or M̅_{w}/M̅ₙ≤1.1.

13. The block copolymer as claimed in claim 11, wherein the block copolymer has a molar mass MW ranging from 4000 g·mol⁻¹≤MW≤40 000 g·mol⁻¹.

14. A pharmaceutical retard system, pharmaceutical administration system with controlled release or pharmaceutical formulation with controlled release comprising one or more block copolymers as claimed in claim 11.

15. The process as claimed in claim 1, wherein the epoxide units are ethylene oxide (EO), propylene oxide (PO), 1-ethoxyethyl glycidyl ether (EEGE) and/or glycidol.

16. The process as claimed in claim 2, wherein said alcohol is selected from the group consisting of methanol, butanol, benzyl alcohol (BnOH), 2-(benzyloxy)ethanol, pentaerythritol, 1,1,1-trimethylolpropane (TMP), bisphenol A, CH₃(CH₂)ₜOH and OH(CH₂)ₜOH where t=1-21.

17. The process as claimed in claim 8, wherein the counterion is selected from the group consisting of potassium, lithium and sodium.

18. The block copolymer as claimed in claim 11, wherein
the residual group of the polyether the blocks Bᵢ is a residual of a polyether selected from the group consisting of polyethylene oxide (PEO), polypropylene oxide (PPO), polyethoxyethylene glycidyl ether (PEEGE), linear or branched polyglycidol (PG, hbPG) and the copolymer epoxide units of the blocks Bᵢ are selected from the group consisting of ethylene oxide (EO), propylene oxide (PO), 1-ethoxyethyl glycidyl ether (EEGE) and/or glycidol;
the residual group of the polyether of the blocks Cᵢ is a residual of a polyether selected from the group consisting of polyethylene oxide (PEO), polypropylene oxide (PPO), polyethoxyethylene glycidyl ether (PEEGE), linear or branched polyglycidol (PG, hbPG); and
the residual group of the polyether of I is a residual group of a polyether selected from polyethylene oxide (PEO), polypropylene oxide (PPO), polyethoxyethylene glycidyl ether (PEEGE), linear or branched polyglycidol (PG, hbPG), monomethyl polyethylene oxide (mPEO), monomethyl propylene oxide (mPPO), monobutyl propylene oxide (mPBO), and the copolymer epoxide units of I are selected from the group consisting of ethylene oxide (EO), propylene oxide (PO), 1-ethoxyethyl glycidyl ether (EEGE) and glycidol, and the residual group of an alcohol of I is selected from the group consisting of methanol, butanol, benzyl alcohol (BnOH), 2-(benzyloxy)ethanol, pentaerythritol, 1,1,1-trimethylolpropane (TMP), bisphenol A, CH₃(CH₂)ₜOH or OH(CH₂)ₜOH where t=1-21.
